(19) United States
(12) Patent Application Publication
(10) Pub. No.: US 2008/0314115 A1
(43) Pub. Date: Dec. 25, 2008

(54) Compliance Apparatus

(75) Inventors: George Charles Faulder, Staffordshire (GB); Richard Martin Faulder, Derbyshire (GB)

Correspondence Address: KING & SCHICKLI, PLLC, 247 NORTH BROADWAY, LEXINGTON, KY, 40507, US

(73) Assignee: Zenics Limited, Leek (GB)

(21) Appl. No.: 11/908,976

(22) PCT Filed: Mar. 15, 2006
(86) PCT No.: PCT/GB2006/000913

§ 371 Date: Sep. 18, 2007

Related U.S. Application Data
(60) Provisional application No. 60/662,983, filed on Mar. 18, 2005.

(30) Foreign Application Priority Data
Mar. 18, 2005 (GB) 0505473.9

(51) Int. Cl. G01N 33/497

(52) U.S. Cl. 73/23.3

(57) ABSTRACT

The invention relates to methods and apparatus for checking compliance with disulfiram prescription regime. The apparatus includes a device for monitoring, on a regular basis the levels of volatiles in the users breath and a comparator for comparing the test results with anticipated results and producing a non-compliance output when a non-compliance event is detected. The apparatus includes an indicator for providing an indication that carbon disulphide is present in the measured volatiles. The detector of the apparatus detects, at least acetone and carbon disulphide.

This invention relates to apparatus for checking compliance with a disulfiram prescription regime.

As is explained in European Patent 0767910, it is possible to monitor the compliance of an alcohol abuser with a disulfiram prescription regime by taking breath readings and comparing the results with anticipated results.

At the time of that Patent, it was thought that the predominant volatile component measured by a process, such as photoionisation, was carbon disulphide, which was created a metabolite of disulfiram.

From one aspect the invention consists in apparatus for checking compliance with a disulfiram prescription regime including a device for monitoring on a regular basis the level of volatiles in a user's breath and a comparator for comparing the test results with anticipated results and producing a non compliance output when a non compliance event is detected characterised in that the apparatus includes an indicator for providing an indication that carbon disulphide is present in the measured volatiles.

What the applicants have subsequently discovered is that in fact a major volatile in the breath of a patient on a disulfiram prescription regime is acetone, because the disulfiram interferes with the metabolism of acetone leading to a significant increase in the acetone in the patient's breath. Accordingly non compliance would show a dip in this acetone level.

It might be thought therefore that it would simply suffice to measure the acetone levels in a patient's breath and indeed that this would be desirable, because the changes in acetone level are more dramatic that those of carbon disulphide. However, the applicants have appreciated that there are other causes of changes in the acetone level of a patient. For example untreated diabetes will give rise to a higher acetone reading as will the participation on a fat burning diet such as the ATKINS™ diet.

In the case of diabetes, it is particularly important that the change in acetone level should not be misinterpreted, because disulfiram is contra indicated for diabetics. However, diabetes is a disease which can occur at any time in life and so somebody may become diabetic after their prescription regime has been introduced.

The applicants have therefore appreciated that whilst it is beneficial to measure the level of acetone in order to benefit from the substantial changes which exist between compliance at with the prescription regime and non compliance, it is essential to check for the presence of carbon disulphide as a confirmation that the change in acetone level is due to non compliance, rather than some other change in lifestyle or health.

As indicated above, the sensor will detect acetone. The applicants have determined that, surprisingly, the ratio of carbon disulphide to acetone varies between individuals and is consistent over time. Accordingly, at a first level at least, this ratio can be used to ascertain that there is at least a substantial likelihood that the person giving a sample into the machine is the person who is intended to be monitored. Thus the apparatus may include a calculator for computing if carbon disulphide/acetone ratio and a comparator for comparing the ratio with a stored ratio and for indicating if the compared ratios are not at least substantially equal.

Additionally or alternatively the apparatus may include a camera or the like linked to a visual recognition system so that again the identity of the user can be checked. In its simplest form the camera may simply provide a photograph or real time image for an operator to monitor. More usually the signal will be passed through a visual recognition system software and produce an indication of whether or not the user is the intended user. As indicated the visual recognition system may be remotely located. In any event the system may be provided with telemetry means allowing for communication of results and/or indications to a remote location.

From another aspect the invention consists in a method of checking compliance with a disulfiram prescription regime, including

(a) giving a loading dose to a patient;
(b) obtaining an indication of the carbon disulphide present in the loaded patient's breath;
(c) subsequently obtaining an indication of the carbon disulphide in a patient's breath after a maintenance dose of disulfiram should have been taken;
(d) comparing the indications; and
(e) determining non-compliance if indications vary more than ±50%.

Although the invention has been defined above it is to be understood it includes any inventive combination of the features set out above or in the following description.

The invention may be performed in various ways and a specific embodiment will now be described by way of example with reference to the accompanying drawing in which:

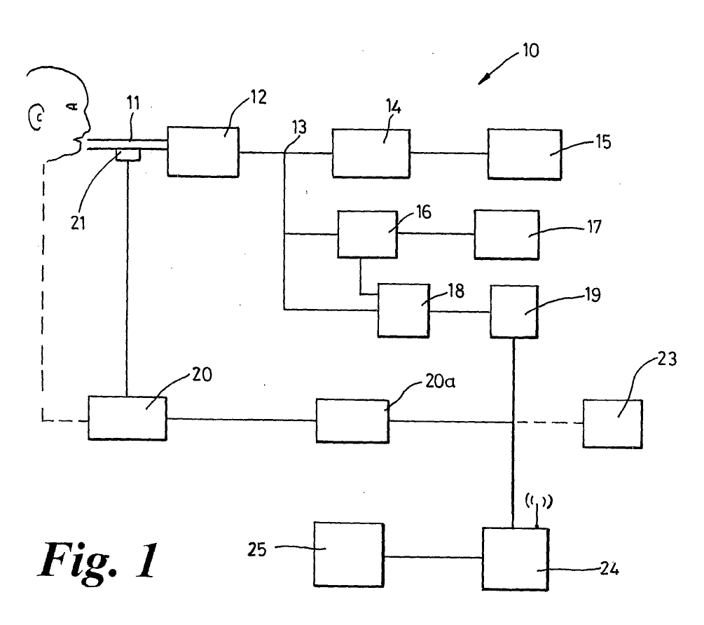

FIG. 1 is a diagrammatic representation of the apparatus;

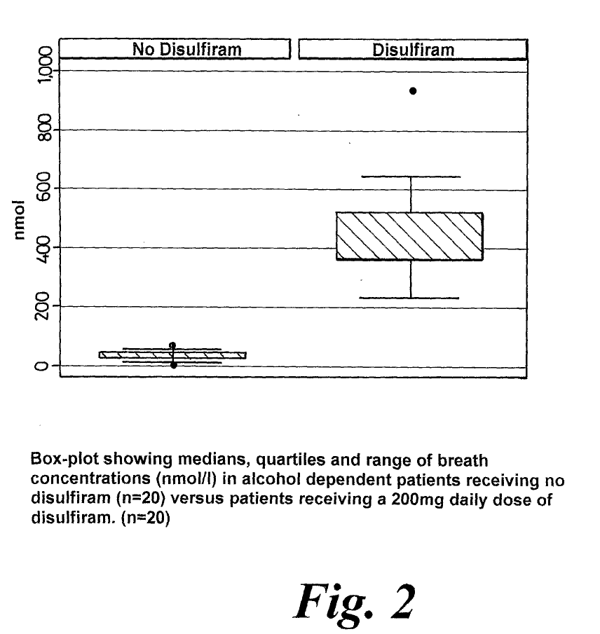

FIG. 2 is a plot of test results from study 1 (see below); and

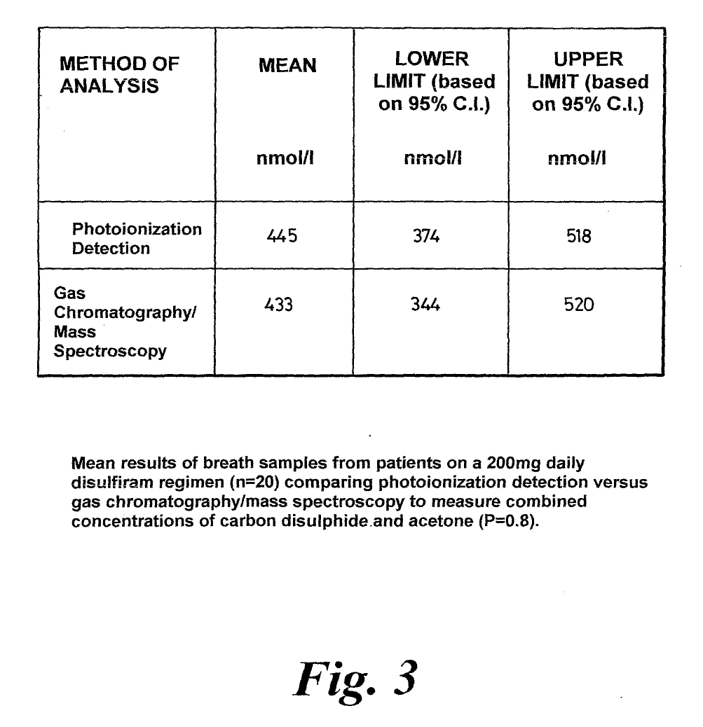

FIGS. 3 to 5 are tables of the results from the described studies.

Compliance apparatus 10 having a breath input 11 to a sensor 12 that produces an output at 13 representing a measure of the volatiles in the user's breath. In the simplest form of the apparatus 10 the signal 13 is fed to a comparator 14, which compares the output with a predetermined value to produce a signal on compliance indicator 15 indicating whether or not the prescription regime is being complied with. However, for the reasons indicated above, such an apparatus could give a false output, if acetone is being produced by the user for some other reason and so in accordance with one aspect of the present invention the signal on 13 is also fed to a filter 16 which strips out that part of the signal arising from carbon disulphide and feeds it to a carbon disulphide indicator 17 to show whether or not carbon disulphide is present. The output of filter 16 and the signal on 13 can also be fed to a ratio computer 18. This will initially subtract the signal produced by 16 from the signal on 13 to provide an acetone value and then compute the ratio of carbon disulphide to acetone and feed this to an identity indicator 19, which compares the ratio with a previously stored value produced by the intended user and produces an indication to show whether or not the intended user is using the apparatus 10.

A camera or the like 20 can also be provided for taking an image of the user whilst breath is flowing into the input 11, which can be determined by pressure sensor 21, and the output from the camera 20 is fed to a visual recognition system 20a that determines whether the user is the intended user and feeds an indication of this to either the indicator 19 or a separate indicator 23. It will be appreciated that one could utilise either the visual recognition system or the ratio system in any particular piece of apparatus, but it is preferred that both are used, because then the statistical chances of a false identity indicator being generated are then extremely small.

The apparatus 10 may also include a transmitter 24 for transmitting the indication at indicator 19 to a remote monitoring unit. A GPS unit 25 may also be connected to the transmitter 24 so that the users whereabouts can be determined.

### EXAMPLES

### Study 1

Forty patients attending the Drug and Alcohol Unit at Shelton Hospital, Shrewsbury, who had previously been diagnosed alcohol dependent and who had given informed consent, were asked to blow into a breath analyser fitted with a disposable mouthpiece. Twenty inpatients were treated with the following supervised disulfiram regimen; loading dose: day 1, 800 mg, day 2, 600 mg, day 3, 400 mg followed by daily maintenance doses of 200 mg. Breath samples were collected throughout each day during the maintenance dose period. Multiple breath samples were obtained from a further twenty alcohol dependent patients who were not receiving disulfiram treatment. The age, gender, ethnic origin, body mass index and smoker/non smoker details were recorded for each patient.

A total of 489 breath test results were used in this study. A mean breath result was calculated for each patient. A comparison between means and variation between groups was made using t-tests and one-way ANOVA respectively.

Breath samples were obtained and analysed by the hand-held breath analyser for the combined concentration of carbon disulphide and acetone, which is known to increase significantly in patients treated with disulfiram.

The method of analysis used to determine the combined carbon disulphide and acetone concentrations in the breath was photoionization detection. The concentrations in breath of carbon disulphide and acetone rise and fall rapidly after each dose. For example, carbon disulphide reaches peak plasma concentrations 5 to 6 hours after dosing and has a breath elimination half-life of 13.3 hours.

The results of the 489 breath tests provided the data from which two reference ranges were established, one for patients known to be on a daily dose of 200 mg of disulfiram and one for patients not on disulfiram treatment. These ranges are suggested as a guide for the expected test results for patients who are compliant with maintenance daily disulfiram treatment. Their sensitivity and specificity were tested in study 2.

Twenty separate samples of breath from patients attending the Shelton Clinic and receiving a daily maintenance dose of 200 mg of disulfiram were sent to The Health and Safety Laboratory, a World Health Organisation collaborating centre, for independent analysis by gas chromatography/mass spectroscopy. An independent samples t-test was performed to compare the mean results of those who had been breath analysed by photoionization and those who had had their breath analysed by gas chromatography/mass spectroscopy.

### Study 2

Patients attending the Alcohol Problems Clinic in Edinburgh who had given informed consent were asked to provide breath samples. Some patients were receiving a supervised disulfiram regimen (meaning that the drug was taken dispersed in 50 ml of water under the view of a nurse) consisting of Monday, 400 mg, Wednesday, 400 mg, Friday, 600 mg; the remainder were not receiving disulfiram therapy. The investigators were to be blind to the patients' treatment regimen. Patients were to be in ‘steady state’, having either received their loading dose or to have been on the supervised regimen and taking disulfiram for at least the previous week.

Breath samples were taken from groups of these patients at post-dose intervals of three days, two days and one day. The two gases, carbon disulphide and acetone, produced in breath as a result of disulfiram therapy, rise and fall rapidly after each dose. The prolonged time intervals of three and two days post dose therefore presented the opportunity to test the breath analyser to the limits of detectable disulfiram breath metabolites.

The investigator conducting the breath testing procedures in Edinburgh did not know which patients were on disulfiram treatment and which were not. Breath test results were recorded for each coded patient and data were sent to Shelton Hospital where, on the basis of the breath test results alone, the Edinburgh patients were categorised as either on disulfiram treatment or not on disulfiram treatment. The reports were then returned to the Edinburgh clinic for comparison with the known disulfiram status of each patient to calculate the sensitivity and specificity of the breath analyser for the three day, two day and one day time intervals.

### Results

### Study 1

FIG. 2 shows that in 489 breath tests obtained from alcohol dependent patients, combined carbon disulphide/acetone breath concentrations were significantly higher in the twenty who were on a 200 mg daily maintenance dose of disulfiram than the twenty who were not receiving disulfiram treatment. The range for patients on disulfiram treatment was 374 nmol/l to 518 nmol/l (95% C.I.) with a mean value of 445 nmol/l. The range for patients not on disulfiram treatment was found to be 27 nmol/l to 40 nmol/l (95% C.I.) with a mean value of 33 nmol/l (P<0.001).

Bartlett's test for variance within the two groups showed a normal distribution (F-test=<0.001 and P=<0.001). There was a significant difference in the variation between the mean of the two groups (P=<0.001).

Twenty breath samples from the group receiving daily maintenance doses of 200 mg of disulfiram were sent to The Health and Safety Laboratory for analysis of carbon disulphide and acetone by gas chromatography/mass spectroscopy. A comparison is given in FIG. 3. There was no significant difference between the analytical methods (P=0.8).

### Study 2

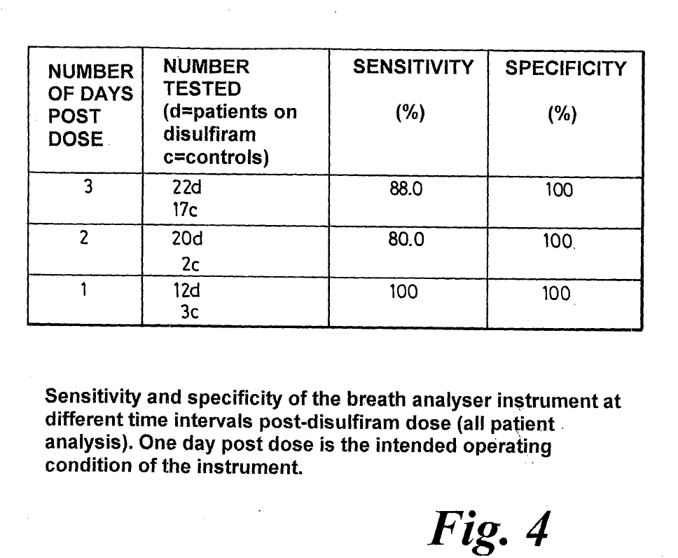

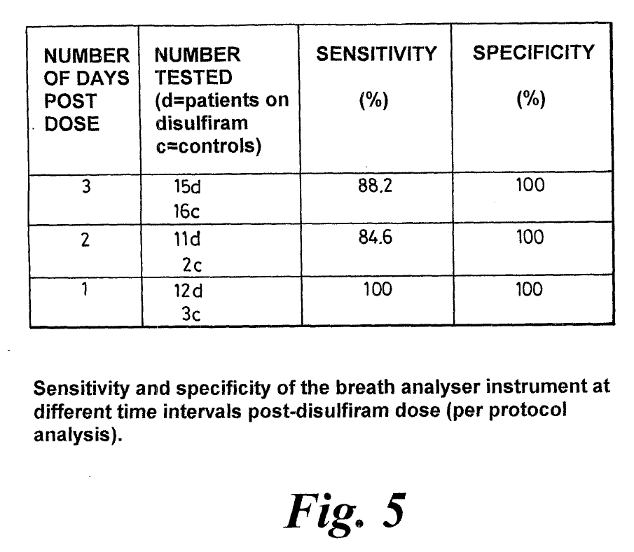

Breath sample data taken from patients attending the Alcohol Problems Clinic in Edinburgh are given in the tables in FIGS. 4 and 5.

A total of thirteen patients were should have been excluded from the study, according to the protocol, for the following reasons: three patients were known to the investigator who was therefore no longer ‘blind’, three patients had only just completed their loading doses and were not yet on maintenance treatment and seven patients had not complied with their prescribed disulfiram dose regimen during the previous week, so were not at a steady-state. The main analysis has excluded the data for these patients (‘per protocol’ analysis). However, all patients were included in the ‘all patient’ analysis shown in the Table of FIG. 4.

The mean breath test result of the Edinburgh patients on thrice weekly disulfiram doses were significantly lower, 121 nmol/l, compared to those of the Shelton patients, 445 nmol/l, on daily disulfiram doses. This was a reflection of the different steady-state levels between the two different regimens employed. The known half-life periods of disulfiram and its metabolites would predict this observation. Consequently Edinburgh patient results that were above the Shelton Clinic “no disulfiram” reference range of 40 nmol/l (FIG. 2) were scored as positive for disulfiram. This method of assessment produced no false positives (specificity of 100% for three day, two day and one day intervals). However, there were false negatives at the three day and the two day interval (sensitivities 88.2%, and 84.6% respectively for the per protocol sample). Sensitivity at an interval of one day was 100%.

These studies will be reported in the Journal Addiction.

Thus, the hand-held breath analyser can, within ten seconds, distinguish between patients who are compliant with a daily maintenance dose of disulfiram and those who, for whatever reason, are not. In its simplest form the invention therefore includes determining a patient's carbon disulphide plus acetone output after a loading dose and then comparing that output with subsequent measurements.

Variance will indicate non-compliance. It would appear that a variation of more than ±50% is sufficient to show non compliance.

1. Apparatus for checking compliance with a disulfiram prescription regime including a device for monitoring on a regular basis the level of volatiles in a user's breath and a comparator for comparing the test results with anticipated results and producing a non-compliance output when a non-compliance event is detected characterised in that the apparatus includes an indicator for providing in indication that carbon disulphide is present in the measured volatiles.

2. Apparatus as claimed in claim 1 wherein the detector detects at least acetone and carbon disulphide.

3. Apparatus as claimed in claim 1 wherein the detector is a photoionisation detector.

4. Apparatus as claimed in claim 1 wherein the indicator includes an analyser for identifying the presence of carbon disulphide in test results.

5. Apparatus as claimed in claim 4 wherein the indication is audible, visual and/or graphic.

6. Apparatus as claimed in claim 1 wherein acetone is a monitored volatile.

7. Apparatus as claimed in claim 6 including a calculator for computing a carbon disulphide/acetone ratio and a comparator for comparing the ratio with a stored ratio and for indicating if the compared ratios are not at least substantially equal.

8. Apparatus as claimed in claim 1 including a visual system for capturing an image of a user.

9. Apparatus as claimed in claim 8 included a visual recognition system for checking that the user is an intended user.

10. Apparatus as claimed in claim 9 wherein the visual recognition system is remotely located.

11. Apparatus as claimed in claim 1 including telemetry means for communicating results and/or indications to a remote location.

12. Apparatus as claimed in claim 1 including recording the indication after a loading dose and means for comparing each later indication with the recorded indication.

13. Apparatus as claimed in claim 12 further including means for calculating a mean from a series of indications and therein the mean is recorded as the recorded indication.

14. Apparatus as claimed in claim 1 further including a global positioning system unit.

15. A method of checking compliance with a disulfiram prescription regime, including
(a) giving a loading dose to a patient;
(b) recording an indication of the carbon disulphide present in the loaded patient's breath;
(c) subsequently obtaining an indication of the carbon disulphide in a patient's breath after a maintenance dose of disulfiram should have been taken;
(d) comparing the indications; and
(e) determining non-compliance if indications may be more than ±0.5%.

16. A method as claimed in claim 15 wherein the indications are taken within 24 hours of the time at which the dose should be taken.

17. (canceled)

18. Apparatus as claimed claim 1 including means for notifying a monitoring unit in the event of non-compliance.

19. Apparatus as claimed in claim 1 wherein a non-compliance event is an indication of a concentration of more than 600 nmol/l or less than 200 nmol/l of carbon disulphide in a user's breath.

20. Apparatus as claimed in claim 2 wherein the detector is a photoionisation detector.
